(19) United States
(12) Patent Application Publication
(10) Pub. No.: US 2025/0009727 A1
(43) Pub. Date: Jan. 9, 2025

(54) WATER-SOLUBLE SALTS OF DNA BINDING AGENTS

(71) Applicant: BOARD OF REGENTS, THE UNIVERSITY OF TEXAS SYSTEM, Austin, TX (US)

(72) Inventors: Waldemar PRIEBE, Houston, TX (US); Stanislaw SKORA, Houston, TX (US); Izabela FOKT, Houston, TX (US); Rafal ZIELINSKI, Houston, TX (US)

(73) Assignee: BOARD OF REGENTS, THE UNIVERSITY OF TEXAS SYSTEM, Austin, TX (US)

(21) Appl. No.: 18/710,267

(22) PCT Filed: Nov. 17, 2022
(86) PCT No.: PCT/US2022/080021

§ 371 Date: May 15, 2024

Related U.S. Application Data
(60) Provisional application No. 63/280,531, filed on Nov. 17, 2021.

(51) Int. Cl. A61K 31/4439; A61K 45/06; A61P 35/00; C07D 405/14

(52) U.S. Cl. CPC A61K 31/4439; A61K 45/06; A61P 35/00; C07D 405/14

(57) ABSTRACT

Provided herein are salts which intercalate into the DNA of a cell and are capable of crossing the blood brain barrier of the formula (I) wherein the variables are as defined herein. These salts may be show improved water solubility and/or aqueous stability than free base or other salts of these compounds. Pharmaceutical compositions of the compounds and methods of treating cancer, for example brain, lung, or pancreatic cancer, are also provided herein.

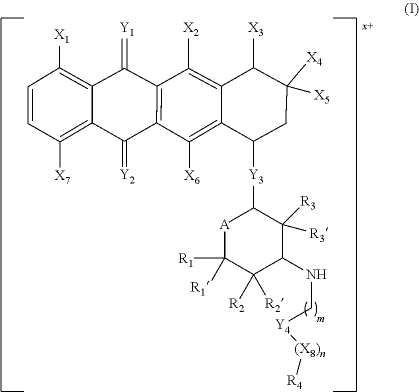

This application claims the benefit of priority to U.S. Provisional Application No. 63/280,531, filed on Nov. 17, 2021, the entire contents of which are hereby incorporated by reference.

### BACKGROUND

### 1. Field

The present disclosure relates generally to the field of medicinal chemistry. More particularly, it concerns salts of chemotherapeutic compounds capable of binding to DNA and/or crossing the blood brain barrier.

### 2. Description of Related Art

The anthracyclines daunorubicin and doxorubicin (DOX) are some of the more commonly used chemotherapeutic antibiotics. The anthracyclines achieve their cytotoxic effect by several mechanisms, including inhibition of topoisomerase II; intercalation between DNA strands, thereby interfering with DNA and RNA synthesis; production of free radicals that react with and damage intracellular proteins and nucleic acids; chelation of divalent cations; and reaction with cell membranes. The wide range of potential sites of action may account for the broad efficacy as well as the toxicity of the anthracyclines (Young et al., 1985). Although there are marked differences in the clinical use of daunorubicin and doxorubicin, their chemical structures differ only by a single hydroxyl group on C14. Previous modifications of these compounds have been undertaken to improve their selectivity towards specific tumors types (U.S. Pat. Nos. 6,673,907, 7,109,177, and 7,557,090 and PCT Publication WO 2008/029294). In particular, brain cancers can be difficult to treat as many compounds cannot easily cross the blood brain barrier (Pardridge, 1999; Bickel, et al., 2001).

Previous compounds had shown improved activity including the ability to cross the blood brain barrier. (PCT Publication WO 2017/049091). While these compounds showed improved activity and an ability to pass through the blood brain barrier, these compounds showed limited water solubility.

As such, new derivatives that improve the water solubility of these active compounds are needed.

### SUMMARY

In some aspects, the present disclosure provides a new salt of a DNA binding agent with an anthracycline core and a long amine containing tail. In some aspects, the present disclosure provides salts of the formula:

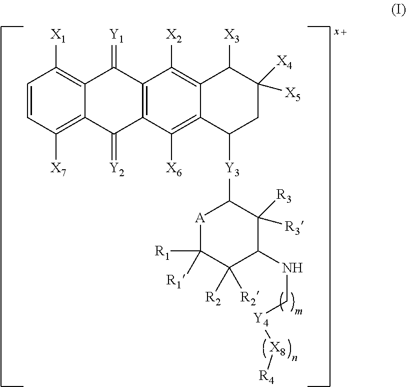

wherein:
X₁, X₂, X₃, X₆, and X₇ are each independently hydrogen, halo, hydroxy, carboxy, ester_{(C≤12)}, substituted ester_{(C≤12)}, alkoxy_{(C≤12)}, or substituted alkoxy_{(C≤12)};
X₄ is acyl_{(C≤18)} or substituted acyl_{(C≤12)};
X₅ is hydrogen, hydroxy, alkoxy_{(C≤12)}, or substituted alkoxy_{(C≤12)};
Y₁, Y₂, and Y₃ are each independently O, S, or NH;
A is O or S;
R₁, R₁′, R₂, R₂′, R₃, and R₃′ are each independently hydrogen, amino, halo, hydroxy, mercapto, or
   alkyl_{(C≤8)}, alkoxy_{(C≤8)}, alkylthio_{(C≤8)}, alkylamino_{(C≤8)}, dialkylamino_{(C≤8)}, or a substituted version of any of these groups;
Y₄ is arenediyl_{(C≤12)} or a substituted version thereof;
each X₈ is independently —X₉-heteroarenediyl_{(C≤12)} or substituted —X₉-heteroarenediylh_{(C≤12)}, wherein:
   X₉ is —NHC(O)— or —C(O)NH—;
R₄ is —X₁₀-heteroaryl_{(C≤12)} or substituted —X₁₀-heteroaryl_{(C≤12)}, wherein:
   X₁₀ is —NHC(O)— or —C(O)NH—;
m is 0, 1, 2, or 3; and
n is 1, 2, or 3,
   or an anion, wherein the anion is not a chloride, provided that the anion can be one or more anions such that the total charge of the anion balances the charge of the cation, x.

In some embodiments, the anion is selected from a halide except chloride, hydroxide, phosphate, sulfate, a thiolate containing compound, a sulfinate containing compound, a sulfate containing compound, or a carboxylate containing compound.

In some embodiments, the anion is a compound of the formula:

⁻OS(O)ₐR₅ (II)

wherein:
a is 0, 1, or 2; and
R₅ is alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups.

In other embodiments, the anion is a compound of the formula:

⁻OC(O)R₆ (III)

wherein:
R₆ is alkyl_{(C≤30)}, cycloalkyl_{(C≤8)}, alkenyl_{(C≤30)}, aryl_{(C≤8)}, heteroaryl_{(C≤8)}, heterocycloalkyl_{(C≤8)}, or a substituted version of any of these groups.

In some embodiments, the anion is citrate, tartrate, hippurate, lactate, camsylate, acetate, phosphate, fumarate, maleate, sulfate, succinate, mesylate, tosylate, gluconate, glucuronate, malate, benzoate, besylate, isethionate, lauryl sulfate, napsylate, oleate, pamoate, bromide, iodide, or nitrate.

In some embodiments, the salts are further defined by the formula:

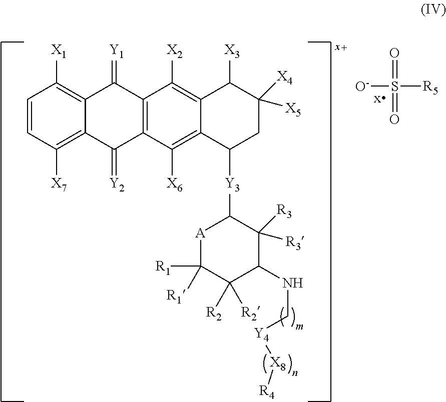

wherein:
X₁, X₂, X₃, X₆, and X₇ are each independently hydrogen, halo, hydroxy, carboxy, ester_{(C≤12)}, substituted ester_{(C≤12)}, alkoxy_{(C≤12)}, or substituted alkoxy_{(C≤12)};
X₄ is acyl_{(C≤18)} or substituted acyl_{(C≤18)};
X₅ is hydrogen, hydroxy, alkoxy_{(C≤12)}, or substituted alkoxy_{(C≤12)};
Y₁, Y₂, and Y₃ are each independently O, S, or NH;
A is O or S;
R₁, R₁′, R₂, R₂′, R₃, and R₃′ are each independently hydrogen, amino, halo, hydroxy, mercapto, or
   alkyl_{(C≤8)}, alkoxy_{(C≤8)}, alkylthio_{(C≤8)}, alkylamino_{(C≤8)}, dialkylamino_{(C≤8)}, or a substituted version of any of these groups;
Y₄ is arenediyl_{(C≤12)} or a substituted version thereof;
each X₈ is independently X₉ heteroarenediyl_{(C≤12)} or substituted —X₉-heteroarenediyl_{(C≤12)}, wherein:
   X₉ is —NHC(O)— or —C(O)NH—;
R₄ is —X₁₀-heteroaryl_{(C≤12)} or substituted —X₁₀-heteroaryl_{(C≤12)}, wherein:
   X₁₀ is —NHC(O)— or —C(O)NH—;
R₅ is alkyl_{(C≤12)}, aryl_{(C≤12)}, or a substituted version of either group;
m is 0, 1, 2, or 3; and
n is 1, 2, or 3.

In some embodiments, the salts are further defined as:

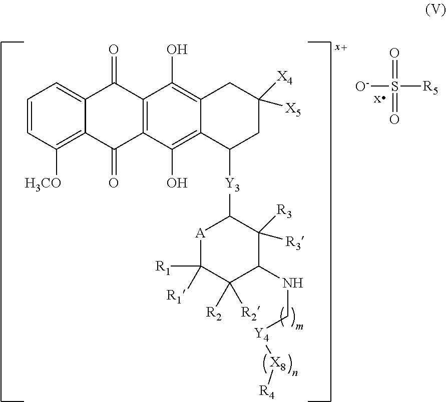

wherein:
X₄ is acyl_{(C≤18)} or substituted acyl_{(C≤18)};
X₅ is hydrogen, hydroxy, alkoxy_{(C≤12)}, or substituted alkoxy_{(C≤12)};
Y₃ is O, S, or NH;
A is O or S;
R₁, R₁′, R₂, R₂′, R₃, and R₃′ are each independently hydrogen, amino, halo, hydroxy, mercapto, or alkyl_{(C≤8)}, alkoxy_{(C≤8)}, alkylthio_{(C≤8)}, alkylamino_{(C≤8)}, dialkylamino_{(C≤8)}, or a substituted version of any of these groups;
Y₄ is arenediyl_{(C≤12)}, heteroarenediyl_{(C≤12)}, or a substituted version of either of these groups;
each X₈ is independently —X₉-heteroarenediyl_{(C≤12)} or substituted —X₉-heteroarenediyl_{(C≤12)}, wherein:
   X₉ is —NHC(O)— or —C(O)NH—;
R₄ is —X₁₀-heteroaryl_{(C≤12)} or substituted —X₁₀-heteroaryl_{(C≤12)}, wherein:
   X₁₀ is —NHC(O)— or —C(O)NH—;
R₅ is alkyl_{(C≤12)}, aryl_{(C≤12)}, or a substituted version of either group;
m is 0, 1, 2, or 3; and
n is 1, 2, or 3.

In some embodiments, the salts are further defined as:

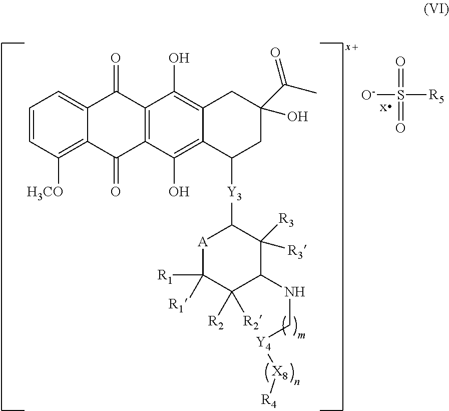

wherein:
R₁, R₁′, R₂, R₂′, R₃, and R₃′ are each independently hydrogen, amino, halo, hydroxy, mercapto, or
   alkyl_{(C≤8)}, alkoxy_{(C≤8)}, alkylthio_{(C≤8)}, alkylamino_{(C≤8)}, dialkylamino_{(C≤8)}, or a substituted version of any of these groups;
Y₄ is a covalent bond, arenediyl_{(C≤12)}, heteroarenediyl_{(C≤12)}, or a substituted version of either of these groups;
each X₈ is independently X₉ heteroarenediyl_{(C≤12)} or substituted —X₉-heteroarenediyl_{(C≤12)}, wherein:
   X₉ is —NHC(O)— or —C(O)NH—;
R₄ is —X₁₀-heteroaryl_{(C≤12)} or substituted —X₁₀-heteroaryl_{(C≤12)}, wherein:
   X₁₀ is —NHC(O)— or —C(O)NH—;
R₅ is alkyl_{(C≤12)}, aryl_{(C≤12)}, or a substituted version of either group;
m is 0, 1, 2, or 3; and
n is 1, 2, or 3.

In some embodiments, the salts are further defined as:

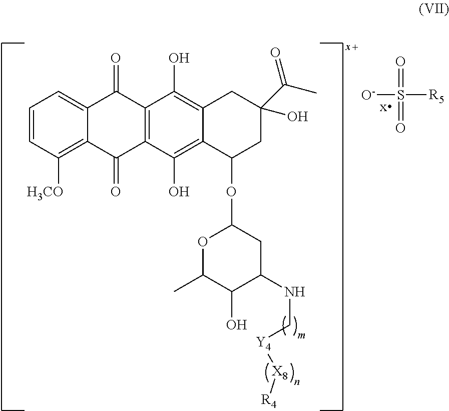

wherein:
Y₄ is a covalent bond, arenediyl_{(C≤12)}, heteroarenediyl_{(C≤12)}, or a substituted version of either of these groups;
each X₈ is independently —X₉-heteroarenediyl_{(C≤12)} or substituted —X₉-heteroarenediyl_{(C≤12)}, wherein:
   X₉ is —NHC(O)— or —C(O)NH—;
R₄ is —X₁₀-heteroaryl_{(C≤12)} or substituted —X₁₀-heteroaryl_{(C≤12)}, wherein:
   X₁₀ is —NHC(O)— or —C(O)NH—;
R₅ is alkyl_{(C≤12)}, aryl_{(C≤12)}, or a substituted version of either group;
m is 0, 1, 2, or 3; and
n is 1, 2, or 3.

In some embodiments, X₇ is alkoxy_{(C≤12)} or substituted alkoxy_{(C≤12)} such as methoxy. In other embodiments, X₇ is halo such as fluoro.

In some embodiments, X₄ is acyl_{(C≤18)} or substituted acyl_{(C≤18)}. In some embodiments, X₄ is acyl_{(C≤8)} such as —C(O)CH₃. In other embodiments, X₄ is substituted acyl_{(C≤8)} such as —C(O)CH₂OH.

In some embodiments, R₁ is alkyl_{(C≤8)} such as methyl. In some embodiments, R₂ is hydroxy. In some embodiments, m is 1. In some embodiments, Y₄ is arenediyl_{(C≤12)} such as benzenediyl.

In some embodiments, X₈ is —X₉-heteroarenediyl_{(C≤12)}. In some embodiments, X₈ is —NHC(O)-heteroarenediyl_{(C≤12)}. In some embodiments, the heteroarenediyl_{(C≤12)} of X₈ is 2,4-pyrroldiyl or 2,4-N-methylpyrroldiyl. In some embodiments, X₅ is:

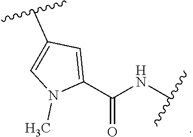 [SMILES: *NC(=O)c1cc(*)cn1C]

In some embodiments, R₄ is —NHC(O)-heteroaryl_{(C≤12)} or substituted —NHC(O)-heteroaryl_{(C≤12)}. In some embodiments, the heteroarenediyl_{(C≤12)} of R₄ is 2-pyridinyl.

In some embodiments, R₅ is alkyl_{(C≤12)} or substituted alkyl_{(C≤12)}. In some embodiments, R₅ is alkyl_{(C≤12)} such as methyl. In other embodiments, R₅ is aryl_{(C≤12)} or substituted aryl_{(C≤12)}. In some embodiments, R₅ is aryl_{(C≤12)} such as phenyl or tolyl.

In some embodiments, the salts are further defined as:

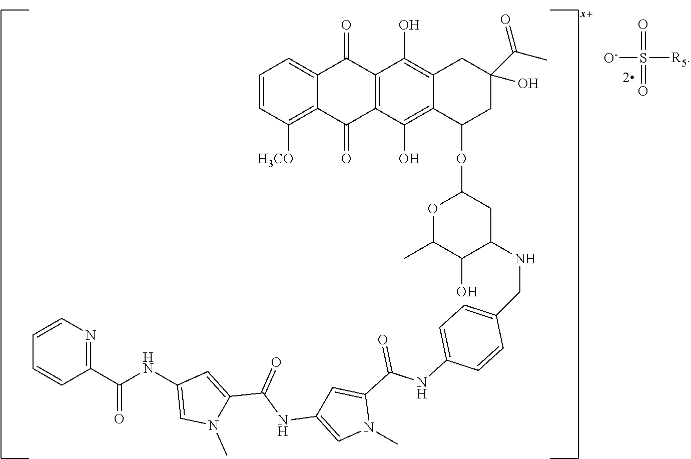

In some embodiments the salts are further defined as:

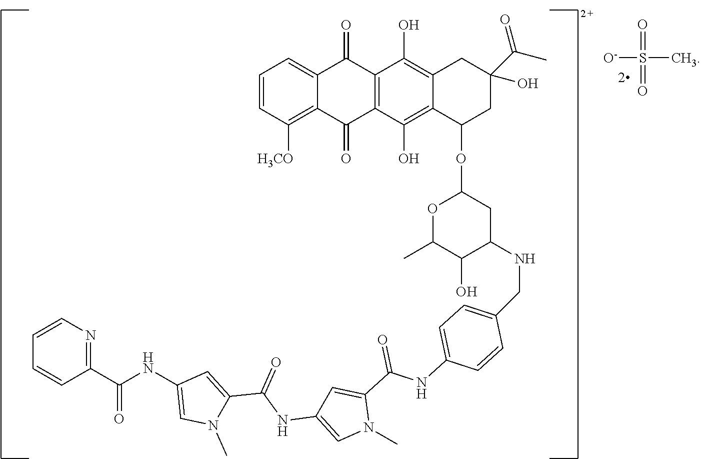

In some embodiments, the salts are further defined as:

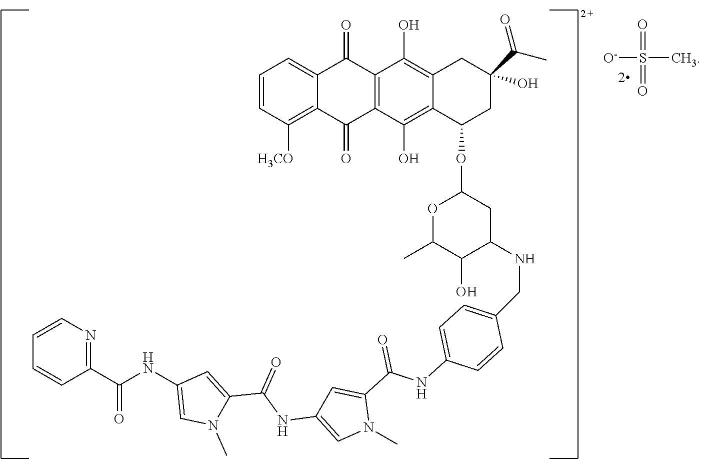

In another aspect, the present disclosure provides pharmaceutical compositions comprising:
(a) a salt described herein; and
(b) a pharmaceutically acceptable carrier.

In some embodiments, the pharmaceutical compositions are formulated for intraarterial, intravenous, or oral administration. In some embodiments, the pharmaceutical compositions are formulated as a unit dose.

In still yet another aspect, the present disclosure provides methods of treating cancer in a patient comprising administering a therapeutically effective amount of a salt or composition described herein to the patient in need thereof.

In some embodiments, the cancer is a cancer of the of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is lung cancer, brain cancer, or pancreatic cancer. In some embodiments, the cancer is brain cancer. In other embodiments, the cancer is glioblastoma. In other embodiments, the cancer is a metastasis to the brain. In other embodiments, the cancer is a metastasis to the brain by melanoma, lymphoma, breast, or lung cancer.

In some embodiments, the salt or composition crosses the blood-brain barrier. In some embodiments, the methods comprise administering the salt systemically and allowing the salt to penetrate the brain by diffusion across the blood brain barrier.

In some embodiments, the methods comprise administering a second anti-cancer therapy. In some embodiments, the second anti-cancer therapy is a second chemotherapeutic compound, radiation therapy, surgery, or immunotherapy. In some embodiments, the patient is a mammal such as a human.

In some embodiments, the methods comprise administering the salt once. In other embodiments, the methods comprise administering the salt two or more times.

In yet another aspect, the present disclosure provides salts or compositions described herein for use in the preparation of a medicament for the treatment of cancer.

In still yet another aspect, the present disclosure provides uses of a salt or composition described herein in the manufacture for a medicament for the treatment of cancer.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Structure and DNA intercalation model for WP1244.

FIG. 2—In vivo efficacy of WP1244 in orthotopic U87 GBM model. Eighteen nude athymic mice were implanted intracranially with 0.5 mln U87 GBM cells. Animals were randomized into three experimental groups (n=6). The treatment was initiated on day 10 and consisted of three doses ow WP1244 at 1 and 5 mg/kg given IP.

FIG. 3—In vitro efficacy of WP1874 in ependymoma, normal kidney, glioblastoma, and medulloblastoma cell lines.

FIGS. 4A-B—Subcellular distribution of WP1874 and induction of DNA damage. (FIG. 4A) BT58 cells were exposed to WP1874 or doxorubicin at 10 μM for 90 min. The subcellular distribution was assessed using confocal imaging; (FIG. 4B) Induction of DNA damage was assessed using pH2Ag as a biomarker for double strand breaks by Western Blot.

FIG. 5—Pharmacokinetic and brain uptake of WP1874 in mice. CD-1 mice received single IV bolus injection of WP1874 (10 mg/kg). The animals were euthanized at indicated timepoints followed by determination of WP1874 in plasma and brain using LC/MS/MS.

FIG. 6—Single- and multiple-dose toxicity of WP1874 in CD-1 mice. CD-1 mice received single or multiple (8 weekly injections) of WP1874 at 10-2.5 mg/kg. Mice were monitored for 40 days (single dose) and 53 days for multiple dose experiments.

### DESCRIPTION OF ILLUSTRATIVE EMBODIMENTS

The present disclosure provides salts that show improved water solubility of a compound which may be used to treat cancer and/or bind to DNA. Without wishing to be bound by any theory, it is believed that the anthracycline moiety intercalates into the DNA helix and the polyamine tail binds the phosphate backbone. In some embodiments, the salts described herein shows increased permeability across the blood brain barrier allowing the salts to penetrate into brain tissue while having improved water solubility than simple chloride salts of the compounds. The salts provided herein may be used to treat cancer especially cancers of the brain, lungs, and pancreas.

### I. SALTS OF THE PRESENT DISCLOSURE

The salts provided by the present disclosure is shown above in the summary section and in the claims below. The salts may be referred to in the context of this disclosure as either a salt or a compound. It may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The compounds described herein may be a salt of a compound, wherein the anion of the salt is not a chloride. In some embodiments, the anion may comprise a sulfate group such as an anion of formula II. In some embodiments, the anion is further defined by the formula:

⁻OS(O)ₐR₅ (II)

wherein:
a is 0, 1, or 2; and
R₅ is alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups.

In other embodiments, the anion may comprise a carboxylate group such as anion of formula III. In some embodiments, the anion is further defined as:

⁻OC(O)R₆ (III)

wherein:
R₆ is alkyl_{(C≤30)}, cycloalkyl_{(C≤8)}, alkenyl_{(C≤30)}, aryl_{(C≤8)}, heteroaryl_{(C≤8)}, heterocycloalkyl_{(C≤8)}, or a substituted version of any of these groups.

Some non-limiting examples of possible anions that may be used in the present disclosure include citrate, tartrate, hippurate, lactate, camsylate, acetate, phosphate, fumarate, maleate, sulfate, succinate, mesylate, tosylate, gluconate, glucuronate, malate, benzoate, besylate, isethionate, lauryl sulfate, napsylate, oleate, pamoate, bromide, iodide, or nitrate.

The compound of the disclosure may contain one or more asymmetrically-substituted carbon or nitrogen atoms, and may be isolated in optically active or racemic form.

Thus, all chiral, diastereomeric, racemic form, epimeric form, and all geometric isomeric forms of a chemical formula are intended, unless the specific stereochemistry or isomeric form is specifically indicated. The compound may occur as racemates and racemic mixtures, single enantiomers, diastereomeric mixtures and individual diastereomers. In some embodiments, a single diastereomer is obtained. The chiral centers of the compound of the present disclosure can have the S or the R configuration.

Chemical formulas used to represent the compound of the disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compound of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The compound of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

The compound of the disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise. In particular, the compounds described herein may be salts that show improved water solubility and/or improved aqueous stability. Furthermore, the compounds described herein may be salts that show improved manufacturability relative to the free base or the chloride salts.

The compound of the present disclosure may also exist in prodrug form. Since prodrugs are known to enhance numerous desirable qualities of pharmaceuticals (e.g., solubility, bioavailability, manufacturing, etc.), the compound employed in some methods of the invention may, if desired, be delivered in prodrug form. Thus, the disclosure contemplates prodrugs of the compound of the present disclosure as well as methods of delivering prodrugs. Prodrugs of the compound employed in the disclosure may be prepared by modifying functional groups present in the compound in such a way that the modifications are cleaved, either in routine manipulation or in vivo, to the parent compound. Accordingly, prodrugs include, for example, the compound described herein in which a hydroxy, amino, or carboxy group is bonded to any group that, when the prodrug is administered to a subject, cleaves to form a hydroxy, amino, or carboxylic acid, respectively.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compound provided herein, including any solvates thereof are within the scope of the present disclosure.

### II. CANCER AND OTHER HYPERPROLIFERATIVE DISEASES

While hyperproliferative diseases can be associated with any disease which causes a cell to begin to reproduce uncontrollably, the prototypical example is cancer. One of the key elements of cancer is that the cell's normal apoptotic cycle is interrupted and thus agents that decreases cell counts may be used as therapeutic agents for treating these diseases. In this disclosure, the compound described herein may be used to kill or inhibit the growth of a cancer cell (e.g., leading to decreased cancer cell counts) or a hyperproliferative cell and may be used to treat a variety of cancers including cancers of the brain, lungs, and pancreas. In some embodiments, the compound may be used to treat brain cancer such as a glioma.

Cancer cells that may be treated with the compound of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### III. PHARMACEUTICAL COMPOSITIONS AND THERAPEUTIC ADMINISTRATION

### A. Pharmaceutical Compositions and Preparations

Where clinical applications are contemplated, it will be necessary to prepare pharmaceutical compositions in a form appropriate for the intended application. In some embodiments, such formulation with the compound of the present disclosure is contemplated. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the salts, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compounds of the present disclosure, its use in therapeutic compositions is contemplated. Supplementary active ingredients also can be incorporated into the compositions.

The salts of the present disclosure may include classic pharmaceutical preparations. Administration of these salts according to the present disclosure will be via any common route so long as the target tissue is available via that route. Such routes include oral, nasal, buccal, rectal, vaginal, urethral, or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The salts may also be administered parenterally or intraperitoneally. Solutions of the active salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the salts in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the salts described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### B. Methods of Treatment

In particular, the compositions that may be used in treating cancer in a patient (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the cancer and other drugs being administered concurrently. A composition as described herein is typically administered at a dosage that induces death of cancerous cells (e.g., induces apoptosis of a cancer cell), as assayed by identifying a reduction in hematological parameters (complete blood count—CBC), or cancer cell growth or proliferation. In some embodiments, amounts of the compounds used to treat the cancer is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the compounds may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

### IV. COMBINATION THERAPIES

It is envisioned that the compound described herein may be used in combination therapies with an additional chemotherapeutic agent or a compound which mitigates one or more of the side effects experienced by the patient.

Furthermore, it is very common in the field of cancer therapy to combine therapeutic modalities. The following is a general discussion of therapies that may be used in conjunction with the therapies of the present disclosure.

To treat cancers using the methods and compositions of the present disclosure, one would generally contact a tumor cell or subject with a compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the compound described herein may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. Additionally, the combination therapy may comprise treating the patient with the compounds provided herein and either radiotherapy or surgery. Other combination therapies may include the compounds provided herein and one or more additional chemotherapeutic compounds. A general discussion of potential chemotherapeutic co-therapies is included below.

### A. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBJ-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above. In some embodiments, the chemotherapeutic agent is a chemotherapeutic drug which inhibits one or more kinases which is misregulated or overexpressed in a cancer. In some embodiments, the chemotherapeutic drug is afatinib, aflibercept, axitinib, bevacizumab, bosutinib, cabozantinib, cetuximab, crizotinib, dasatinib, erlotinib, forstamatinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, trastuzumab, tofacitinib, vandetanib, or vemurafenib.

### V. DEFINITIONS

When used in the context of a chemical group: “hydrogen” means —H; “hydroxy” means —OH; “oxo” means=O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanate” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof, “mercapto” means —SH; and “thio” means=S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “=” means a double bond, and “≡” means triple bond. The symbol “ [symbol] ” represents an optional bond, which if present is either single or double. The symbol “ [symbol] ” represents a single bond or a double bond. Thus, for example, the formula

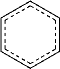

includes

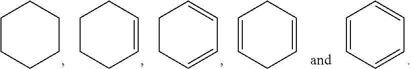

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

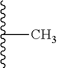

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

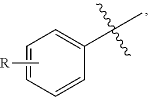

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

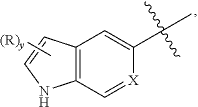

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ⁱBu), and —CH₂C(CH₃)₃(neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: =CH₂, =CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

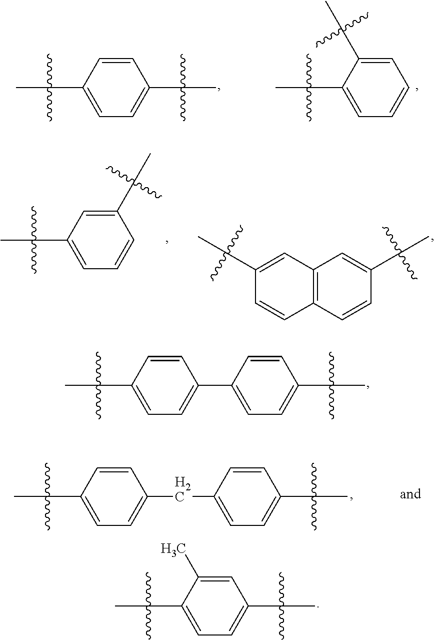

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

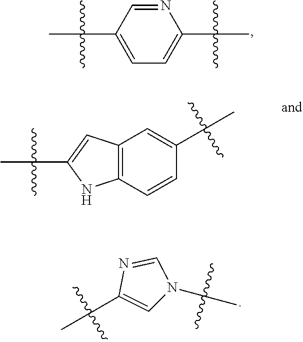 [SMILES: *c1ccc(*)nc1 ; *c1ccc2[nH]c(*)cc2c1 ; *c1cn(*)cn1]

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C6H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. The term “ester” refers to the group —C(O)R, in which R is an alkoxy. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃(tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects. If none of these values apply, then the term means+5% of the stated value.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “patient” or “subject” refers to a living mammalian organism, such as a human, monkey, horse, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Structure and Features of WP1244

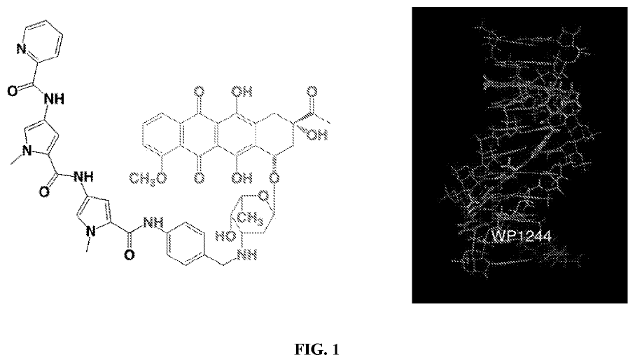

In an approach that combines DNA intercalating and DNA “minor-groove-binding” modules, compounds that potentially bind up to 10 bp long sequences of DNA were developed. Compound WP1244 was isolated and tested as a hydrochloride salt. The structure of compound WP1244 and a model of its binding mode are presented in FIG. 1.

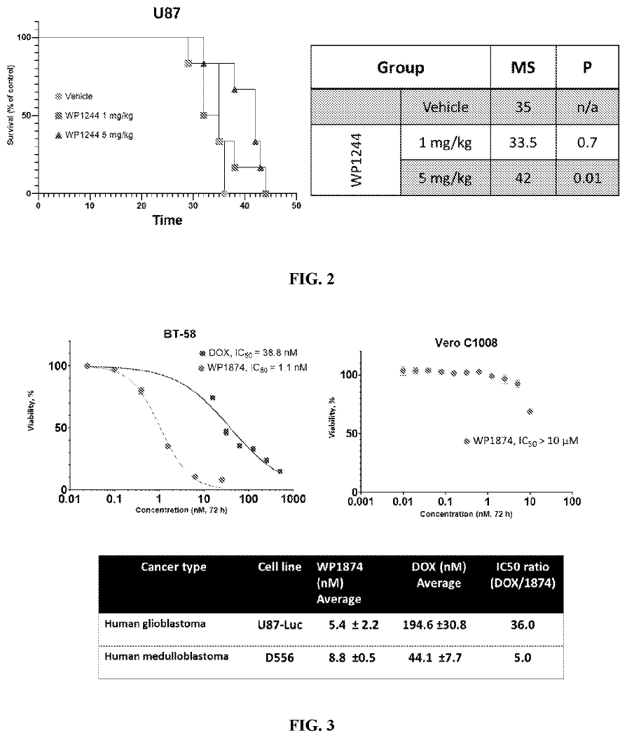

WP1244 was observed to have an especially high CNS uptake combined with the picomolar to low nanomolar cytotoxicity against ependymoma and glioblastoma multiforme (GBM) cell lines and demonstrated in vivo activity in the orthotopic U87 model of GBM (see FIG. 2).

### Example 2—Evaluation of Solubility of WP1244 Salts

Progress was limited by the poor solubility of WP1244 hydrochloride salt. To improve solubility and develop IV injectable formulation, different salts of WP1244 were prepared. WP1874, a mesylate salt of WP1244, was shown to exhibit unexpectedly good solubility (see Table 1 below).

TABLE 1
Solubility of WP1244 and WP1874 in Water
and 5% Dextrose at room temperature.

| | H₂O | 5% Dextrose |
|---|---|---|
| WP1244 | Lower than 0.25 mg/mL | Lower than 0.25 mg/mL |
| WP1874 | 2.6 mg/mL | 2.5 mg/mL |

### Example 3—In Vitro Efficacy of WP1874

WP1874, similarly to its parental compound, showed high cytotoxicity in ependymoma, GBM, and medulloblastoma cell lines with IC₅₀ in low nanomolar range. Interestingly, WP1874 appeared to be not cytotoxic against normal kidney cells (Vero C1008) with IC₅₀>10 μM (see FIG. 3).

### Example 4—WP1874 Subcellular Distribution, and DNA Damage

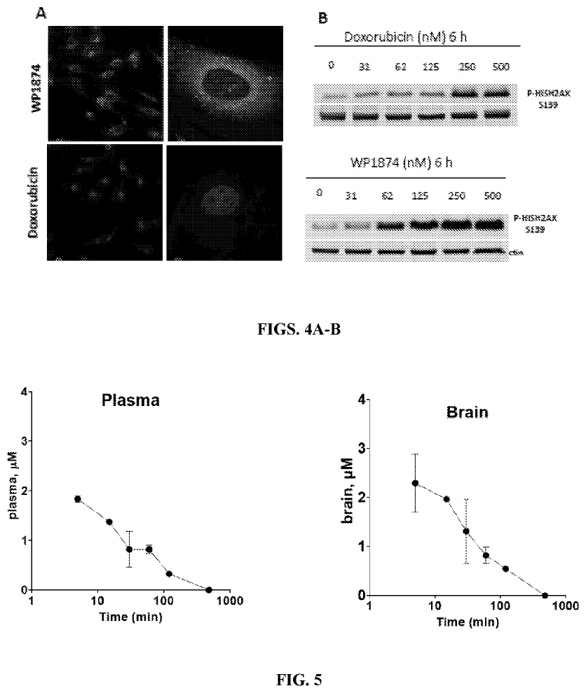

Fluorescent imaging of BT58 ependymoma cells revealed distinct subcellular distribution pattern of WP1874 when compared to doxorubicin (see FIG. 4A). Furthermore, WP1874 exposure was shown to lead to extensive DNA damage (see FIG. 4B).

### Example 5—Pharmacokinetic and CNS Penetration of WP1874

Preliminary pharmacokinetic and biodistribution studies were performed in CD-1 mice with intact brain, revealing enhanced penetration of WP1874 to the brain with Cmax values approximately 1.5-fold greater in the brain (2.3 μg/g) than in plasma (1.8 ug/ml). (see FIG. 5).

### Example 6—Toxicity of WP1874

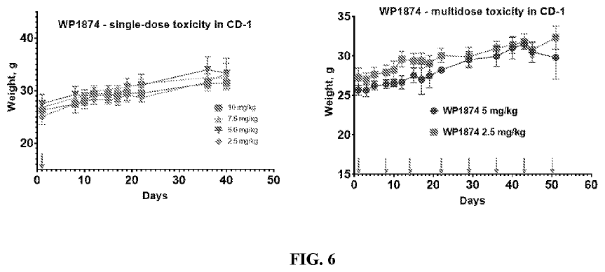

Acute toxicity experiments of WP1874 administrated by IV determined that value of LD₅₀ stayed in 10-15 mg/kg range. No mortalities or any apparent toxicity symptoms were recorded for CD-1, BALB/c and nude athymic mice that received six weekly doses of WP1874 at 2.5 or 5 mg/kg. given intravenously. Intraperitoneal administration was well tolerated up to 5 mg/kg given three times a week for four cycles. (see FIG. 6).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 6,673,907
U.S. Pat. No. 7,109,177
U.S. Pat. No. 7,557,090
PCT Publication WO 2008/029294
PCT Publication WO 2017/049091
Bickel et al., Adv. Drug Deliv. Rev., 46: 247-279, 2001.
Handbook of Pharmaceutical Salts: Properties, and Use, Stahl and Wermuth Eds.), Verlag Helvetica Chimica Acta, 2002.
March's Advanced Organic Chemistry: Reactions, Mechanisms, and Structure, 2007.
Pardridge, J. Neurovirol., 5: 556-569, 1999.
Young et al., N. Engl. J Med., 312:692, 1985.

What is claimed is:

1. A salt comprising a cation of the formula: 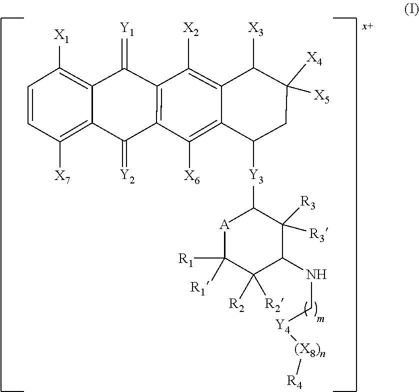
wherein:
X₁, X₂, X₃, X₆, and X₇ are each independently hydrogen, halo, hydroxy, carboxy, ester_{(C≤12)}, substituted ester_{(C≤12)}, alkoxy_{(C≤12)}, or substituted alkoxy_{(C≤12)};
X₄ is acyl_{(C≤18)} or substituted acyl_{(C≤18)};
X₅ is hydrogen, hydroxy, alkoxy_{(C≤12)}, or substituted alkoxy_{(C≤12)};
Y₁, Y₂, and Y₃ are each independently O, S, or NH;
A is O or S;
R₁, R₁′, R₂, R₂′, R₃, and R₃′ are each independently hydrogen, amino, halo, hydroxy, mercapto, or
alkyl_{(C≤8)}, alkoxy_{(C≤8)}, alkylthio_{(C≤8)}, alkylamino_{(C≤8)}, dialkylamino_{(C≤8)}, or a substituted version of any of these groups;
Y₄ is arenediyl_{(C≤12)} or a substituted version thereof;
each X₈ is independently —X₉-heteroarenediyl_{(C≤12)} or substituted —X₉-heteroarenediyl_{(C≤12)}, wherein:
X₉ is —NHC(O)— or —C(O)NH—;
R₄ is —X₁₀-heteroaryl_{(C≤12)} or substituted —X₁₀-heteroaryl_{(C≤12)}, wherein:
X₁₀ is —NHC(O)— or —C(O)NH—;
m is 0, 1, 2, or 3; and
n is 1, 2, or 3,
or an anion, wherein the anion is not a chloride, provided that the anion can be one or more anions such that the total charge of the anion balances the charge of the cation, x.

2. The salt of claim 1, wherein the anion is selected from a halide except chloride, hydroxide, phosphate, sulfate, a thiolate containing compound, a sulfinate containing compound, a sulfate containing compound, or a carboxylate containing compound.

3. The salt of either claim 1 or claim 2, wherein the anion is a compound of the formula:
⁻OS(O)ₐR₅ (II)
wherein:
a is 0, 1, or 2; and
R₅ is alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups.

4. The salt according to any one of claims 1-3, wherein the anion is a compound of the formula:
⁻OC(O)R₆ (III)
wherein:
R₆ is alkyl_{(C≤30)}, cycloalkyl_{(C≤8)}, alkenyl_{(C≤30)}, aryl_{(C≤8)}, heteroaryl_{(C≤8)}, heterocycloalkyl_{(C≤8)}, or a substituted version of any of these groups.

5. The salt according to any one of claims 1-4, wherein the anion is citrate, tartrate, hippurate, lactate, camsylate, acetate, phosphate, fumarate, maleate, sulfate, succinate, mesylate, tosylate, gluconate, glucuronate, malate, benzoate, besylate, isethionate, lauryl sulfate, napsylate, oleate, pamoate, bromide, iodide, or nitrate.

6. The salt according to any one of claims 1-5 further defined by the formula: 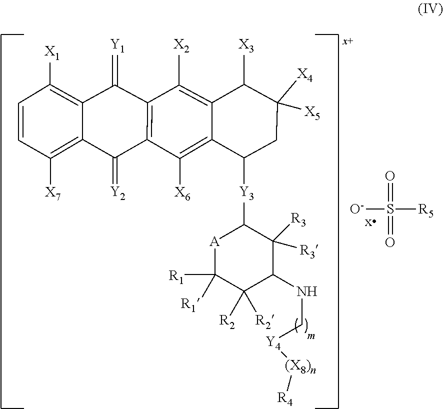
wherein:
X₁, X₂, X₃, X₆, and X₇ are each independently hydrogen, halo, hydroxy, carboxy, ester_{(C≤12)}, substituted ester_{(C≤12)}, alkoxy_{(C≤12)}, or substituted alkoxy_{(C≤12)};
X₄ is acyl_{(C≤18)} or substituted acyl_{(C≤12)};
X₅ is hydrogen, hydroxy, alkoxy_{(C≤12)}, or substituted alkoxy_{(C≤12)};
Y₁, Y₂, and Y₃ are each independently O, S, or NH;
A is O or S;
R₁, R₁′, R₂, R₂′, R₃, and R₃′ are each independently hydrogen, amino, halo, hydroxy, mercapto, or
alkyl_{(C≤8)}, alkoxy_{(C≤8)}, alkylthio_{(C≤8)}, alkylamino_{(C≤8)}, dialkylamino_{(C≤8)}, or a substituted version of any of these groups;
Y₄ is arenediyl_{(C≤12)} or a substituted version thereof,
each X₈ is independently —X₉-heteroarenediyl_{(C≤12)} or substituted —X₉-heteroarenediyl_{(C≤12)}, wherein:
X₉ is —NHC(O)— or —C(O)NH—;
R₄ is —X₁₀-heteroaryl_{(C≤12)} or substituted —X₁₀-heteroaryl_{(C≤12)}, wherein:
X₁₀ is —NHC(O)— or C(O)NH—;
R₅ is alkyl_{(C≤12)}, aryl_{(C≤12)}, or a substituted version of either group;
m is 0, 1, 2, or 3; and
n is 1, 2, or 3.

7. The salt according to any one of claims 1-6 further defined as: 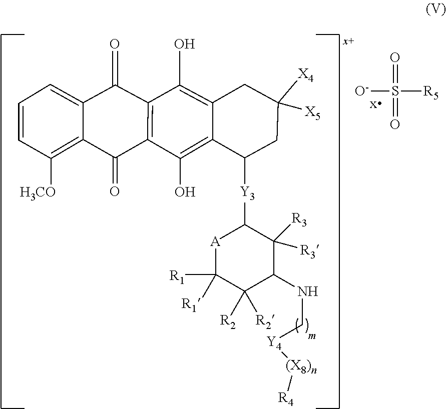
wherein:
X₄ is acyl_{(C≤18)} or substituted acyl_{(C≤18)};
X₅ is hydrogen, hydroxy, alkoxy_{(C≤12)}, or substituted alkoxy_{(C≤12)};
Y₃ is O, S, or NH;
A is O or S;
R₁, R₁′, R₂, R₂′, R₃, and R₃′ are each independently hydrogen, amino, halo, hydroxy, mercapto, or
alkyl_{(C≤8)}, alkoxy_{(C≤8)}, alkylthio_{(C≤8)}, alkylamino_{(C≤8)}, dialkylamino_{(C≤8)}, or a substituted version of any of these groups;
Y₄ is arenediyl_{(C≤12)}, heteroarenediyl_{(C≤12)}, or a substituted version of either of these groups;
each X₈ is independently —X₉-heteroarenediyl_{(C≤12)} or substituted —X₉-heteroarenediyl_{(C≤12)}, wherein:
X₉ is —NHC(O)— or —C(O)NH—;
R₄ is —X₁₀-heteroaryl_{(C≤12)} or substituted —X₁₀-heteroaryl_{(C≤12)}, wherein:
X₁₀ is —NHC(O)— or —C(O)NH—;
R₅ is alkyl_{(C≤12)}, aryl_{(C≤12)}, or a substituted version of either group;
m is 0, 1, 2, or 3; and
n is 1, 2, or 3;
or a pharmaceutically acceptable salt thereof.

8. The salt according to any one of claims 1-7 further defined as: 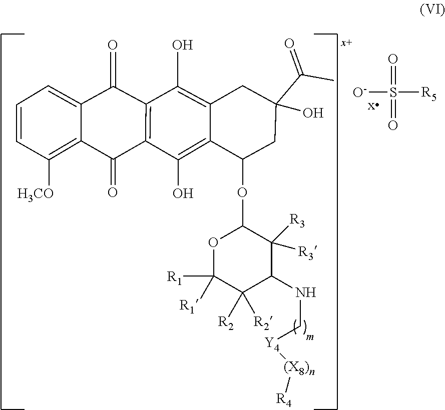
wherein:
R₁, R₁′, R₂, R₂′, R₃, and R₃′ are each independently hydrogen, amino, halo, hydroxy, mercapto, or
alkyl_{(C≤8)}, alkoxy_{(C≤8)}, alkylthio_{(C≤8)}, alkylamino_{(C≤8)}, dialkylamino_{(C≤8)}, or a substituted version of any of these groups;
Y₄ is a covalent bond, arenediyl_{(C≤12)}, heteroarenediyl_{(C≤12)}, or a substituted version of either of these groups;
each X₈ is independently —X₉-heteroarenediyl_{(C≤12)} or substituted —X₉-heteroarenediyl_{(C≤12)}, wherein:
X₉ is —NHC(O)— or —C(O)NH—;
R₄ is —X₁₀-heteroaryl_{(C≤12)} or substituted —X₁₀-heteroaryl_{(C≤12)}, wherein:
X₁₀ is —NHC(O)— or —C(O)NH—;
R₅ is alkyl_{(C≤12)}, aryl_{(C≤12)}, or a substituted version of either group;
m is 0, 1, 2, or 3; and
n is 1, 2, or 3.

9. The salt according to any one of claims 1-8 further defined as: 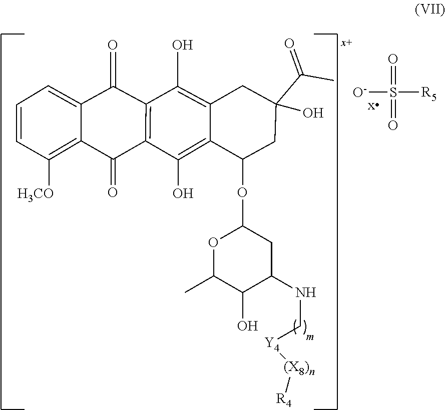
wherein:
Y₄ is a covalent bond, arenediyl_{(C≤12)}, heteroarenediyl_{(C≤12)}, or a substituted version of either of these groups;
each X₈ is independently —X₉-heteroarenediyl_{(C≤12)} or substituted —X₉-heteroarenediyl_{(C≤12)}, wherein:
X₉ is —NHC(O)— or —C(O)NH—;
R₄ is —X₁₀-heteroaryl_{(C≤12)} or substituted —X₁₀-heteroaryl_{(C≤12)}, wherein:
X₁₀ is —NHC(O)— or —C(O)NH—;
R₅ is alkyl_{(C≤12)}, aryl_{(C≤12)}, or a substituted version of either group;
m is 0, 1, 2, or 3; and
n is 1, 2, or 3.

10. The salt according to any one of claims 1-6, wherein X₇ is alkoxy_{(C≤12)} or substituted alkoxy_{(C≤12)}.

11. The salt of claim 10, wherein X₇ is methoxy.

12. The salt according to any one of claims 1-6, wherein X₇ is halo.

13. The salt of claim 12, wherein X₇ is fluoro.

14. The salt according to any one of claims 1-7, wherein X₄ is acyl_{(C≤18)} or substituted acyl_{(C≤18)}.

15. The salt of claim 14, wherein X₄ is acyl_{(C≤8)}.

16. The salt of claim 15, wherein X₄ is —C(O)CH₃.

17. The salt of claim 14, wherein X₄ is substituted acyl_{(C≤8)}.

18. The salt of claim 17, wherein X₄ is —C(O)CH₂OH.

19. The salt according to any one of claims 1-8 and 10-18, wherein R₁ is alkyl_{(C≤8)}.

20. The salt of claim 19, wherein R₁ is methyl.

21. The salt according to any one of claims 1-8 and 10-20, wherein R₂ is hydroxy.

22. The salt according to any one of claims 1-21, wherein m is 1.

23. The salt according to any one of claims 1-22, wherein Y₄ is arenediyl_{(C≤12)}.

24. The salt of claim 23, wherein Y₄ is benzenediyl.

25. The salt according to any one of claims 1-24, wherein X₈ is —X₉-heteroarenediyl_{(C≤12)}.

26. The salt of claim 25, wherein X₈ is —NHC(O)-heteroarenediyl_{(C≤12)}.

27. The salt of claim 26, wherein the heteroarenediyl_{(C≤12)} of X₈ is 2,4-pyrroldiyl or 2,4-N-methylpyrroldiyl.

28. The salt of claim 27, wherein X₈ is:

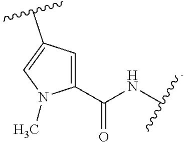 [SMILES: *NC(=O)c1cc(*)cn1C]

29. The salt according to any one of claims 1-28, wherein R₄ is —NHC(O)-heteroaryl_{(C≤12)} or substituted —NHC(O)-heteroaryl_{(C≤12)}.

30. The salt of claim 29, wherein the heteroarenediyl_{(C≤12)} of R₄ is 2-pyridinyl.

31. The salt according to any one of claims 1-28, wherein R₅ is alkyl_{(C≤12)} or substituted alkyl_{(C≤12)}.

32. The salt of claim 31, wherein R₅ is alkyl_{(C≤12)}.

33. The salt of claim 32, wherein R₅ is methyl.

34. The salt according to any one of claims 1-28, wherein R₅ is aryl_{(C≤12)} or substituted aryl_{(C≤12)}.

35. The salt of claim 34, wherein R₅ is aryl_{(C≤12)}.

36. The salt of claim 35, wherein R₅ is phenyl or tolyl.

37. The salt according to any one of claims 1-36, wherein the salt is further defined as:

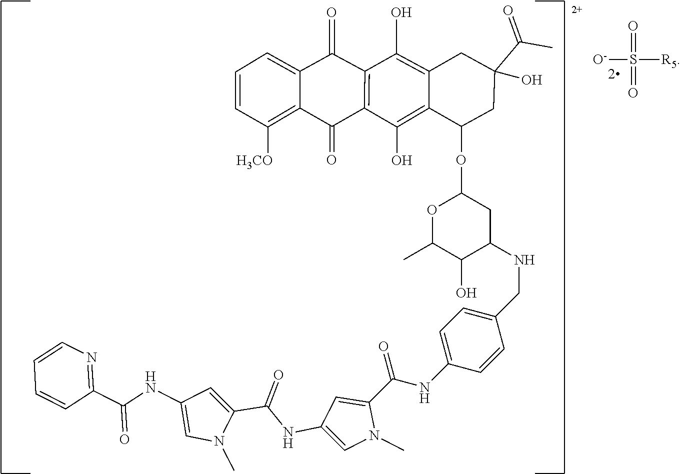

38. The salt according to any one of claims 1-37, wherein the salt is further defined as:

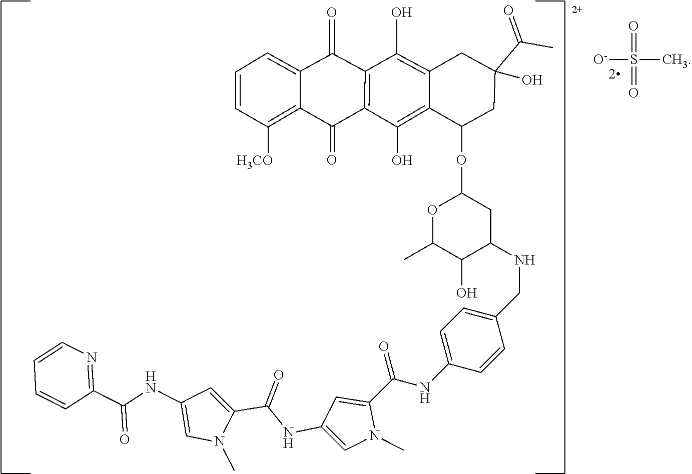

39. The salt according to any one of claims 1-38, wherein the salt is further defined as:

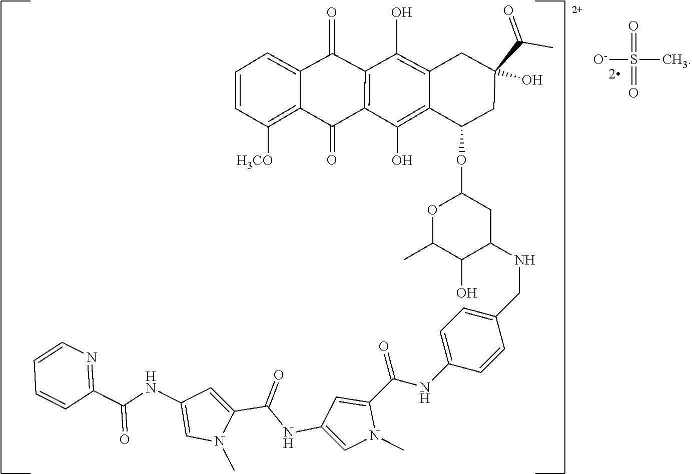

40. A pharmaceutical composition comprising:
(a) a salt according to any one of claims 1-39; and
(b) a pharmaceutically acceptable carrier.

41. The pharmaceutical composition of claim 40, wherein the pharmaceutical composition is formulated for intraarterial, intravenous, or oral administration.

42. The pharmaceutical composition of either claim 40 or claim 41, wherein the pharmaceutical composition is formulated as a unit dose.

43. A method of treating cancer in a patient comprising administering a therapeutically effective amount of a salt or composition according to any one of claims 1-42 to the patient in need thereof.

44. The method of claim 43, wherein the cancer is a cancer of the of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

45. The method of claim 44, wherein the cancer is lung cancer, brain cancer, or pancreatic cancer.

46. The method of claim 45, wherein the cancer is brain cancer.

47. The method of claim 46, wherein the cancer is glioblastoma.

48. The method of claim 46, wherein the cancer is a metastasis to the brain.

49. The method of claim 48, wherein the cancer is a metastasis to the brain by melanoma, lymphoma, breast, or lung cancer.

50. The method according to any one of claims 43-49, wherein the salt or composition crosses the blood-brain barrier.

51. The method of claim 50, wherein the method comprises administering the salt systemically and allowing the salt to penetrate the brain by diffusion across the blood brain barrier.

52. The method according to any one of claims 43-51, wherein the method comprises administering a second anti-cancer therapy.

53. The method of claim 52, wherein the second anti-cancer therapy is a second chemotherapeutic compound, radiation therapy, surgery, or immunotherapy.

54. The method according to any one of claims 43-53, wherein the patient is a mammal.

55. The method of claim 54, wherein the patient is a human.

56. The method according to any one of claims 43-55, wherein the method comprises administering the salt once.

57. The method according to any one of claims 43-55, wherein the method comprises administering the salt two or more times.

58. A salt or composition according to any one of claims 1-42 for use in the preparation of a medicament for the treatment of cancer.

59. Use of a salt or composition according to any one of claims 1-42 in the manufacture for a medicament for the treatment of cancer.
